(19) United States
(12) Patent Application Publication
(10) Pub. No.: US 2014/0235545 A1
(43) Pub. Date: Aug. 21, 2014

(54) The use of HCV immunogenic peptide or a derivative thereof in the prevention or treatment of arthritis

(71) Applicants: YUN CHENG, BEIJING (CN); RUIHE YU, BEIJING (CN); WANZHOU ZHAO, BEIJING (CN)

(72) Inventors: YUN CHENG, BEIJING (CN); RUIHE YU, BEIJING (CN); WANZHOU ZHAO, BEIJING (CN)

(21) Appl. No.: 14/229,686

(22) Filed: Mar. 28, 2014

Related U.S. Application Data
(63) Continuation of application No. PCT/CN2011/080441, filed on Sep. 30, 2011.

(51) Int. Cl. C07K 7/06

(52) U.S. Cl. CPC C07K 7/06
USPC 514/16.7; 530/329

(57) ABSTRACT

The present invention provides the use of hepatitis C virus immunogenic peptides or derivative thereof in the manufacture of a pharmaceutical for preventing or treating arthritis, wherein the peptide or derivative thereof is a peptide represented by formula I or pharmaceutically acceptable salt or ester thereof. The present invention further provides a method for treating arthritis, including administering a pharmaceutical containing therapeutically effective amount of said peptide or derivative thereof to a patient. Xaa1-Gln-Xaa2-Xaa3-Thr-Ser-Gly-Xaa4 (formula I), wherein, Xaa1 is deleted, or Xaa1 is Ala, Gly, Val, Leu or Ile, Xaa2 is Thr or Ser, Xaa3 is Tyr, Phe or Trp, and Xaa4 is deleted, or Xaa4 is Ala, Gly, Val, Leu, Ile or Pro.

### CROSS-REFERENCE TO RELATED APPLICATIONS

This application is a continuation of International Application No. PCT/CN2011/080441 filed on Sep. 30, 2011. The contents of the above identified application are incorporated herein by reference in its entirety.

### FIELD OF THE TECHNOLOGY

The present invention belongs to the technical field of pharmaceutical, and particularly is directed to the use of hepatitis C virus immunogenic peptide or derivative thereof in the manufacture of a pharmaceutical for preventing or treating arthritis, especially combined collagen-adjuvant induced arthritis and adjuvant arthritis.

### BACKGROUND

Arthritis is an inflammatory lesion of joints, a common chronic disease caused by inflammation, infection, trauma, or other factors (such as drugs), and it is mainly shown as red swelling, heat, pain and dysfunction of joints. There are above 100 million people suffering from arthritis in China, and the number are still increasing, where about half of the population that over the age of 50 suffer from arthritis; 90% of females and 80% of males in the population that over the age of 65 suffer from arthritis. Arthritis has become a disease seriously affecting people's daily lives, and even shortens patients' life span by 10 to 15 years in severe cases. Recently, arthritis caused by drugs (e.g, collagen and/or adjuvant, etc.) attracted more and more people's attention, and thus researches on drugs that can effectively treating these types of arthritis has become one of the major research subjects in this field.

CN1194986C and CN1216075C by the present inventor disclose a hepatitis C virus immunogenic peptide or derivative thereof (referred to as 7P peptide or derivative thereof, which is also used in the present invention). The hepatitis C virus immunogenic peptide is an immunogenic peptide that is originally designed according to hepatitis C virus. In these patents, it is proved that 7P peptide or derivative thereof has a function of inducing cytokines r-IFN, IL-4, IL-10 to produce antibody, where r-IFN is secreted by Th1 and is one of the main cytokines of human immune system to fight against viral infections, and it has a considerable significance in removal of HCV (hepatitis C virus), so that 7P peptide or derivative thereof can be used for preventing and/or treating hepatitis C. According to these earlier patents, 7P peptide or derivative thereof may be synthesized by solid phase synthesis or liquid phase synthesis method well-known by those skilled in the art or may be obtained by fusion expression through genetic engineering means and purification, and particularly, the use of peptide having the sequence GQTYTSG and derivative thereof (pharmaceutically acceptable salt or ester thereof) in the prevention and/or treatment of hepatitis C is recorded. Furthermore, PCT/CN2006/001176 by the present inventors discloses the use of 7P peptide or derivative thereof in the prevention and treatment of liver injury, and more specifically, it discloses that 7P peptide or derivative thereof can significantly reduce levels of glutamic-oxaloacetic transaminase and glutamic-pyruvic transaminase in serum, and have significant prevention and treatment effects on immunological liver injury or liver injury caused by liver-toxic chemical substances. Later, CN101559217A by the present inventor further discloses that 7P peptide or derivative thereof also has therapeutic effect on nephritis, and especially, can significantly reduces serum protein-induced nephritis and Heymann's nephritis when the 7P peptide or derivative thereof is administrated through digestive canal.

Arthritis, liver injury and nephritis each has totally different pathogenic mechanisms and totally different clinical manifestation. Surprisingly, in the study, the present inventor found that 7P peptide or derivative thereof can also improve symptoms of arthritis, and especially symptoms of combined collagen-adjuvant induced arthritis and adjuvant arthritis, which are caused by improper use of medicine. There is yet no report in the prior art on the application of the peptide or derivative thereof into prevention and treatment of arthritis.

### SUMMARY

The present invention provides the use of hepatitis C virus immunogenic peptides or derivative thereof in the manufacture of a pharmaceutical for preventing or treating arthritis, providing a new clinical method for the treatment and prevention of arthritic diseases and broadening the potential medicinal fields of 7P peptide.

The present invention further provides a method for treating arthritis by administering a drug comprising a therapeutically effective amount of the hepatitis C virus immunogenic peptide or derivative thereof to a patient. This method can significantly improve arthritis symptoms.

The present invention provides the use of peptide represented by formula I or derivative thereof in the manufacture of a pharmaceutical for preventing or treating arthritis, the derivative including a pharmaceutically acceptable salt or ester of the peptide:

(formula I)
Xaa1-Gln-Xaa2-Xaa3-Thr-Ser-Gly-Xaa4

In which, Xaa1 is deleted, or Xaa1 is Ala, Gly, Val, Leu or Ile,

Xaa2 is Thr or Ser,

Xaa3 is Tyr, Phe or Trp, and

Xaa4 is deleted, or Xaa4 is Ala, Gly, Val, Leu, Ile or Pro.

The inventor's studies have shown that administration of an effective dose of said peptide or derivative thereof is capable of effectively preventing or treating arthritis, especially, combined collagen-adjuvant induced arthritis and adjuvant arthritis. The combined collagen-adjuvant induced arthritis may be induced, for example, by type II collagen in combination with Freund's complete adjuvant, and the adjuvant arthritis may be induced, for example, by Freund's complete adjuvant. The peptide of formula I or derivative thereof has the same basic structure and composition as hepatitis C virus immunogenic peptides or derivative thereof obtained by the inventor in previous studies, and thus is also called as 7P peptide or derivative thereof.

Clinically, combined collagen-adjuvant induced arthritis mainly has joint local heat, swelling, foot volume increase, lameness, skin ulcers and other symptoms, and its pathological manifestations are synovial cells proliferation, the synovial cells appeared to be nodular or clubbing intrude into joint cavity when the proliferation is evident, and congestion of synovial tissue, inflammatory cell infiltration, and other pathological phenomena; adjuvant arthritis mainly has joint local heat, swelling, foot volume increase, lameness and other symptoms, and its pathological manifestations are proliferation, disorganization, or uneven surface of synovial cells, the synovial cells appeared to be nodular or clubbing intrude into joint cavity when the proliferation is evident, and congestion of synovial tissue, inflammatory cell infiltration, and other pathological phenomena. The type II collagen comprises type II collagen from chicken, bovine or rat, which can cause arthritis of rodents and primates.

As used herein, the phrase “pharmaceutically acceptable ester” refers to an ester suitable for contact with human or animal tissue and without excessive toxicity, irritation or allergic reaction. Generally, modification for a peptide by esterification can reduce hydrolysis of the peptide by protease of organisms. The terminal amino, group on a side chain or carboxyl of the peptide of the present invention may be modified to form a pharmaceutically acceptable ester. Modifications for a group on amino acid side chain include, but are not limited to, esterification of a hydroxyl group on side chain of threonine or serine with a carboxylic acid. Preferably, the terminal group of amino acid is protected with a protecting group known for a person skilled in protein chemistry field, such as acetyl, trifluoroacetyl, Fmoc (9-fluorenyl-methoxycarbonyl), Boc(tert-butoxycarbonyl), Alloc (allyloxycarbonyl), C₁₋₃ alkyl, C₆₋₁₂ aralkyl, and the like. Pharmaceutically acceptable esters of 7P peptide are described in detail in PCT/CN2006/001176, related content of which is incorporated herein by reference. In a particular embodiment of the present invention, the inventor has found that the peptide of the present invention can also be used for treating or preventing arthritis under physiological conditions without modifications, and thus it is preferable not to modify N-terminal amino and C-terminal carboxyl of polypeptide of formula I as well as the group on amino acid side chain, i.e., N-terminal chemical group is still as α-amino (—NH₂) of the first amino acid, and C-terminal chemical group is still carboxyl (—COOH) of C-terminal amino acid.

As used herein, the phrase “pharmaceutically acceptable salt” refers to a salt suitable for contact with human or animal tissue and without excessive toxicity, irritation or allergic reaction. The pharmaceutically acceptable salt is well-known in the art. Such a salt can be prepared during the final isolation and purification of the polypeptide of the present invention, or can be prepared separately by the reaction of the peptide with appropriate organic or inorganic acid or base. Representative acid addition salts include, but are not limited to acetate, adipate, alginate, citrate, aspartate, benzoate, benzenesulfonate, bisulfate, butyrate, camphorate, camphorsulfonate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, 3-phenylpropionate, propionate, succinate, tartrate, phosphate, glutamate, bicarbonate, p-toluenesulfonate and undecanoate. Preferred acids that may be used to form pharmaceutically acceptable salts include hydrochloric acid, hydrobromic acid, sulphuric acid, phosphoric acid, oxalic acid, maleic acid, succinic acid and citric acid. For pharmaceutically acceptable base addition salts, the cations include, but are not limited to, alkali metal ion or alkaline earth metal ion, such as lithium, sodium, potassium, calcium and magnesium, quaternary ammonium cation (such as tetramethyl ammonium, tetraethyl ammonium), and cations of ammonium, methylamine, dimethylamine, trimethylamine, triethylamine, diethylamine, ethylamine, diethylamine, ethanolamine, diethanolamine, piperidine, piperazine and the like. Preferred base addition salts include phosphate, tris(hydroxymethyl)aminomethane (tris) and acetate. These salts may generally increase the solubility of the polypeptide, and the salt formed does not substantially alter the activity of the polypeptide.

In summary, according to the present invention, the pharmaceutical to prevent and/or treat arthritis may be the peptide (7P peptide) per se or pharmaceutically acceptable salt or ester thereof.

Furthermore, the peptide or derivative thereof also may be a peptide represented by formula II or pharmaceutically acceptable salt or ester thereof:

(formula II)
Gly-Gln-Thr-Tyr-Thr-Ser-Gly

According to the representation method of amino acids known in this filed, the peptide of formula II may also be abbreviated as GQTYTSG.

In the present invention, for the prevention and/or therapy purposes, suitable formulation of the peptide or derivative thereof may be, for example, in the form of injection, lyophilized powder (for injection), spray, oral solution, oral suspension, tablet, capsule, enteric-coated tablet, pill, powder, granule, sustained-release agent (which can control the active ingredient of the formulation to be slowly released) or controlled release agent (which can control the release of the active ingredient of the formulation), etc. The formulation may include a conventional and pharmaceutically acceptable carrier. The phrase “pharmaceutically acceptable carrier” refers to a non-toxic solid, semi-solid or liquid filler, diluent, adjuvant, coating material or other excipients, such as physiological saline, isotonic glucose solution, buffered saline, glycerol, ethanol, or combinations thereof. In embodiments of the present invention, preferably the pharmaceutical made of the peptide or derivative thereof is administered by injection, and thus, it is preferable to use injection or lyophilized powder for injection after their dissolution in physiological saline as a carrier.

The present invention provides a method for treating arthritis, including administering a pharmaceutical containing therapeutically effective amount of the peptide of formula I or derivative thereof to a patient, where the derivative includes a pharmaceutically acceptable salt or ester of the peptide of formula I.

In a preferred embodiment of the present invention, the pharmaceutical administered contains the peptide of formula II or pharmaceutically acceptable salt or ester thereof as an active ingredient.

A pharmaceutical comprising a therapeutically effective amount of the above peptide or derivative thereof (as active ingredient) can effectively prevent or treat arthritis, especially combined collagen-adjuvant induced arthritis and adjuvant arthritis, where the combined collagen-adjuvant induced arthritis is induced, for example, by type II collagen in combination with Freund's complete adjuvant, and the adjuvant arthritis may be induced, for example, by Freund's complete adjuvant. In one embodiment of the present invention, a pharmaceutical comprising a therapeutically effective amount of 300-3000 μg of the above peptide or derivative thereof is administered to a patient. Preferably, a pharmaceutical comprising a therapeutically effective amount of 480-1800 μg of the above peptide or derivative thereof is administered to a patient. The therapeutically effective amount refers to an effective amount for general adult body weight in a single administration.

In an embodiment of the present invention, preferably, the pharmaceutical comprising a therapeutically effective amount of the above peptide or derivative thereof is administered by injection. Further, the patient is preferably administered in a dose of unit formulation. Here, the unit formulation refers to a formulation that can provide active ingredient in an amount required for a single administration. The unit formulation generally includes, such as, one unit (one piece of) tablet, one unit (one needle of) injection or one unit powder injection, in which the content of active ingredient is an amount required for a single administration. The amount of pharmaceutical required for a single administration to patient may be easily calculated by multiplying patient' body weight by dose per unit of body weight of the patient. For example, during the preparation of pharmaceuticals, adult body weight is generally assumed as 50-70 kg, and using it to calculate the amount of pharmaceutical required for a single administration to patient. Dose per unit body weight of experimental animal and human may be calculated by an equivalent dose conversion relationship. For example, effective dose for human can be derived from the dose for experimental animal according to an equivalent dose conversion relationship between experimental animals and human that well-known for an ordinary skilled in the art (see, for example, guidances of FDA, SFDA and other drug regulatory authorities, or HUANG Ji-han, et al., equivalent dose conversion between different animals and between animal and human in pharmacological experiment, Chinese Journal of Clinical Pharmacology and Therapeutics, 2004 September; 9 (9): 1069-1072). In embodiments of the present invention, it can use body surface area conversion factor 0.018 for human and rat to derive dose for human from rat. In embodiments of the present invention, in order to obtain good treatment results, preferably the peptide or derivative thereof contained in unit formulation is administered to rat at a dose of 50-300 μg/kg rat, more preferably at a dose of 80-180 μg/kg rat, for example, 174 μg/kg rat or 87 μg/kg rat. During the manufacture of pharmaceuticals, manufacturer can get active ingredient content per unit formulation used for human based on the above conversion method. In the technical solutions of the present invention, based on the equivalent dose conversion relationship and general body weight of human, while taking into account pharmaceutical safety, cost and efficiency, the amount of the peptide or derivative thereof contained in unit formulation used for human is preferably 500-3000 μg, more preferably 800-1800 μg, for example, 1740 μg or 870 μg.

According to studies of the present invention, the use of the peptide or derivative thereof for the prevention or treatment of arthritis may effectively improve symptoms of arthritis, in particular to improve symptoms of combined collagen-adjuvant induced arthritis and adjuvant arthritis caused by improper use of pharmaceutical. It can be seen from data from the following embodiments, groups that apply the peptide or derivative thereof (including high, medium and low dose groups) manifest significantly reduced extent of joint swelling and of joint lesion in rat, compared with the model group. Hence, the present invention is contribute to development of pharmaceuticals for arthritis or related disease.

The present invention will be described in details in reference to the following specific embodiments, so as to facilitate understanding the present invention. It should be appreciated that these embodiments are merely illustrative and do not limit the scope of the invention. Various modifications or changes may be made by an ordinary skilled in the art to these embodiments without departing from the scope of the present invention, and these modifications or changes also fall within the scope of the present invention. Furthermore, the present invention refers to literatures published to more clearly describe the present invention, which are incorporated to the present invention in their entireties and constitute a part thereof.

### BRIEF DESCRIPTION OF THE DRAWINGS

FIGS. 1a and 1b show the synovial tissue of rat joint in the control group in Embodiment 1.

FIGS. 2a and 2b show the synovial tissue of rat joint in the model group in Embodiment 1.

FIGS. 3a and 3b show the synovial tissue of rat joint in peptide A low dose group in Embodiment 1.

FIGS. 4a and 4b show the synovial tissue of rat joint in peptide A medium dose group in Embodiment 1.

FIGS. 5a and 5b show the synovial tissue of rat joint in peptide A high dose group in Embodiment 1.

### DETAILED DESCRIPTION

### Embodiment 1

### Protective Effect of Peptide a on Combined Collagen-Adjuvant Induced Arthritis in Rats

1. Testing Material

1.1 Animal

SPF-level SD rats, each weighting 180 g˜220 g, with male and female each in half, provided by Experimental Animal Center of Chinese Academy of Military Medical.

1.2 Pharmaceutical

Peptide with the following sequence is used: GQTYTSG (hereinafter referred to as peptide A), which is synthesized by solid phase peptide synthesis method using 413A automatic peptide synthesizer (available from Perkin Elmer Corporation), the detailed synthetic steps is described in Example 1 of PCT/CN2006/001176. The peptide was to be dissolved in physiological saline when used.

1.3 Grouping and does of Pharmaceutical

SD rats, with male and female each in half, were randomly divided into five groups with 10 rats in each group, respectively are model group (employing physiological saline, calf type II collagen and Freund's complete adjuvant); control group (employing the same volume of physiological saline as the model group); peptide A high, medium and low dose groups (employing calf type II collagen, Freund's complete adjuvant and peptide A, wherein each group is given different doses of peptide A, respectively are 174 μg/kg·d, 87 μg/kg·d and 43.5 μg/kg·d, peptide A is dissolved in physiological saline to form peptide A solution with desired concentration). Peptide A solution in each dose was administered by subcutaneous injection, 0.1 ml of peptide A solution per 100 g body weight of rats. The control group employed the same amount of physiological saline as that of the peptide A solution (0.1 ml of physiological saline per 100 g body weight of rats).

2 Test Methods

2.1 Experimental Scheme

10 mg of calf type II collagen was dissolved into 5 ml 0.1 mol/L of acetic acid solution, and then the resulting solution was placed in a refrigerator at 4° C. overnight, the next day, the solution was mixed with Freund's complete adjuvant in a volume ratio of 1:1, the resulting mixture is sucked and discharged repeatedly by a syringe, until the mixture was emulsified into an emulsion completely. Each group rats were administered according to the following manner. These rats were normally fed every day during the administration.

On the 1st day, except the control group, for rats of other groups (the model group and the peptide A high, medium and low dose groups), 0.5 ml of the emulsion (prepared by calf type II collagen and Freund's complete adjuvant as above) was subcutaneously injected into the base of the tail of every rat, and the injection site is pressed slightly for 30 s so that the emulsion was completely absorbed. On the 8th day, emulsion injection is conducted again in the same way, 0.5 ml for every rat.

From the 2nd day (including this day), the rats of peptide A high, medium and low dose groups each were administered with corresponding dose of peptide A. Then, they were subcutaneously injected with corresponding dose of peptide A for one time every other day, a total of 15 times; at the same time, the rats of the control group and the model group were subcutaneously injected the same amount of physiological saline as that of peptide A for the high, medium and low dose groups every other day, a total of 15 times.

On the 13en day, varying grades of inflammatory reaction were observed from the rats of the model group, manifested as joints local heat, swelling, foot volume increase, lameness, etc. On the 24th day, for most rats of the model group, two rear foot joints and partial forefoot joints reached the greatest degree of swelling, and part of the rats had skin ulcers. Compared with rats of the model group, rats of peptides A high, medium and low dose groups were manifested as significantly reduced joint swelling, and they did not have skin ulcer symptom.

Measurement for the experimental results was conducted as follows. Firstly, before administration to rats of each group, left rear ankle circumference of each rat was measured by a flexible rule as a base value, and then circumference of left rear ankle with inflammation of each rat was measured at the 4th, 8th, 12th, 16th, 20th, 24th, 28th, and 32nd day, respectively. Swelling value obtained by circumference after inflammation minus circumference before inflammation was used to observe changes of primary and secondary inflammation of combined collagen-adjuvant induced rats.

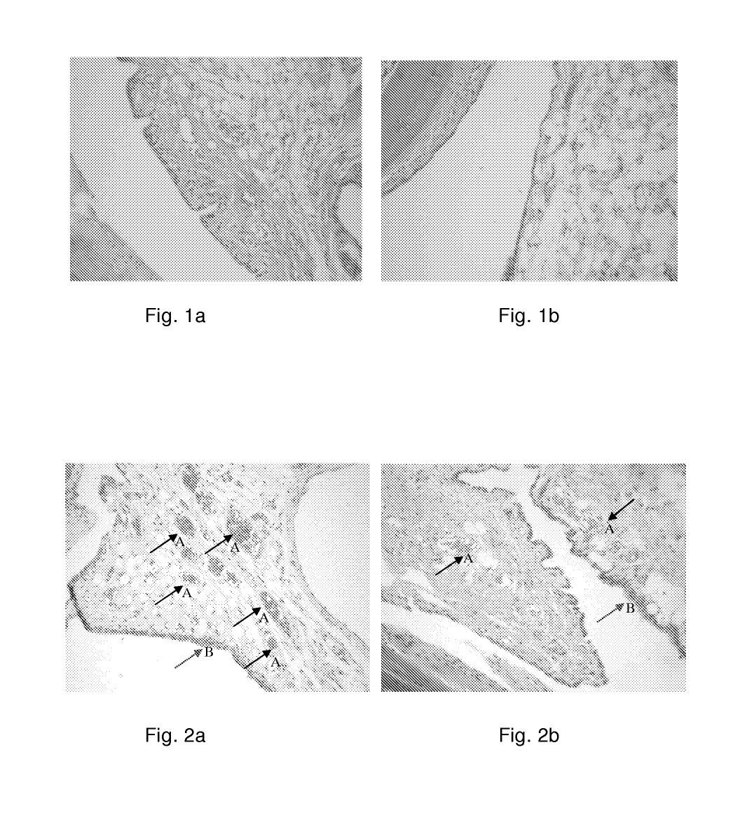

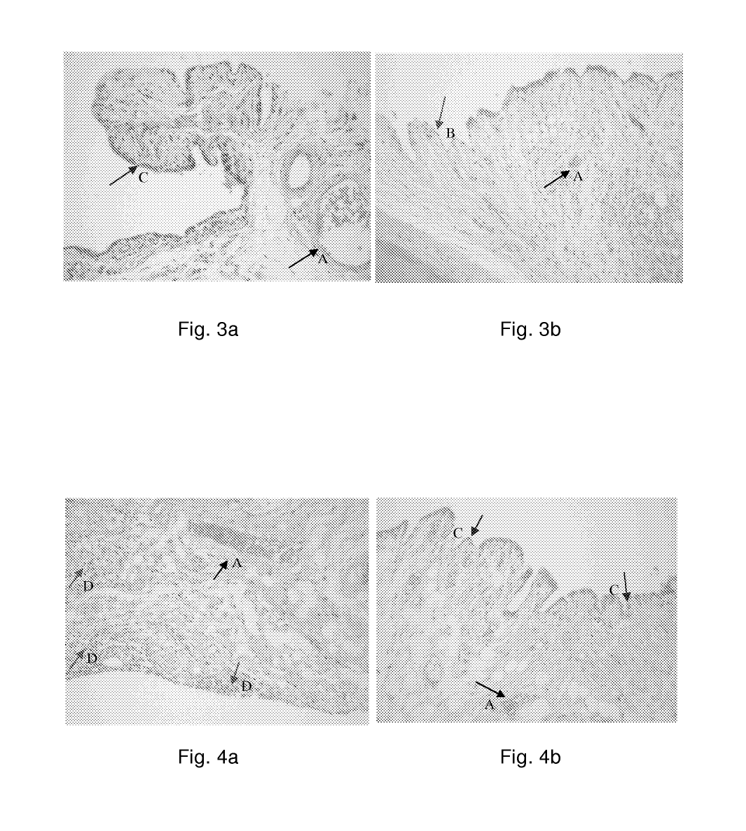

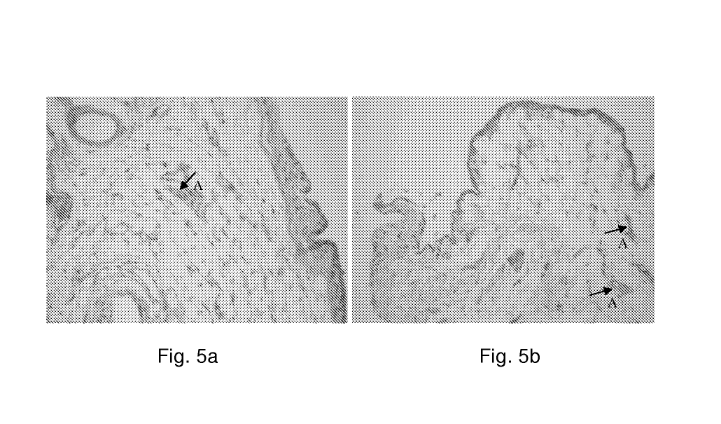

Secondly, at the 32nd day, rats of each group were killed, and their left rear foot ankle joints ware quickly taken out, fixed in 10% formalin and then embedded in paraffin after decalcification, sectioned and stained with HE. The proliferation of synovial tissue, degeneration of covering epithelial cells (synovial cells), congestion of interstitial substance, inflammatory cell infiltration, damage or fibrosis of arthrodial cartilage, and inflammatory cell infiltration or fibrosis of subcutaneous tissue around the joint, were observed. FIGS. 1a-5b were photographs showing different states of synovial tissue of rats of each group observed by using above prepared paraffin sections under ordinary optical microscope at 10× eyepiece. In order to more accurately show the synovial tissue status of rats with different treatment, the inventor took two photographs for rats with different treatment status of each group to illustrate. FIGS. 1a and 1b were photographs showing joint synovial tissue of rats of the control group, from which it can seen that joint synovial tissues of each rat of the control group were manifested as mild congestion, and synovial cells had no obvious degeneration, proliferation and other diseases symptoms. FIGS. 2a and 2b were photographs showing joint synovial tissue of rats of the model group, where black arrow A indicates congestive and expanded blood vessel, and green arrow B indicates degenerated synovial cells. FIG. 2a illustrated moderate congestion and mild proliferation of the synovial tissue as well as mild degeneration of covering synovial cells. FIG. 2b illustrated mild congestion of the synovial tissue, mild degeneration of covering synovial cells and proliferation of local synovial tissue. FIGS. 3a and 3b were photographs showing synovial tissue of rats of peptide A low dose group, where black arrow A indicates congestive and expanded blood vessel, green arrow B indicates degenerated synovial cells, and blue arrow C indicates proliferated synovial tissue. FIG. 3a illustrated mild congestion and significant proliferation and surface projection of the synovial tissue, as well as mild degeneration of covering synovial cells. FIG. 3b illustrated mild congestion and proliferation of the synovial tissue, as well as mild degeneration of the synovial cells. FIG. 4a and FIG. 4b were photographs showing the synovial tissue of rats of peptide A medium dose group, where black arrow A indicates congestive and expanded blood vessel, red arrow D indicates synovial tissue with inflammatory cell infiltration inside thereof, and blue arrow C indicates proliferated synovial tissue. FIG. 4a illustrated mild congestion of local synovial tissue and infiltration of few inflammatory cells. FIG. 4b illustrated mild congestion, uneven surface and mild proliferation of the synovial tissue. FIGS. 5a and 5b were photographs showing the synovial tissue of rats of peptide A high dose group, where black arrow A indicates congestive and expanded blood vessel. FIGS. 5a and 5b illustrated mild congestion of local synovial tissue of the rats, having no significant surface proliferation. According to severity of joint lesions, the lesions were scored as “1 score (few or mild lesion)”, “2 scores (moderate or medium lesion)”, “3 scores (large or severe lesion)”, and “4 scores (very severe lesion)”. Very mild lesion was scored as “0.5”, and no lesion tissue was scored as “0”. Accumulate all scores to obtain a total score for one group, and then calculate average score ( X̅±SD) for each animal in each group. The lower score indicates the milder grade of the lesion.

2.2 Data processing: all of the data were processed, where the pathology score was conducted using rank sum test, and other data was processed using t test, and then the results were statistically analyzed.

3 Results

3.1 Effect of Peptide A on Combined Collagen-Adjuvant Induced Arthritis in Rats

From the data shown in table 1 below that the effect of peptide A in different doses on ankle joint swelling value (mm) and inhibition rate (%) ( X̅±SD, n=10) of rats with combined collagen-adjuvant induced arthritis, it can be seen that calf type II collagen in combination with Freund's complete adjuvant can cause significant joint swelling in rats. Compared to rats of the model group, rats of peptides A high, medium and low dose groups had significantly reduced joint inflammation reaction induced by the calf type II collagen in combination with Freund's complete adjuvant (*P<0.05 or **P<0.01), manifested as significantly reduced joint swelling value, where inhibition rate to the joint swelling in peptide A high dose group is nearly 45%.

TABLE 1

| | Dose | The 4^{th} | The 8^{th} | The 12^{th} | The 16^{th} | The 20^{th} | The 24^{th} | The 28^{th} | The 32^{nd} |
|---|---|---|---|---|---|---|---|---|---|
| Group | (μg/kg · d) | day | day | day | day | day | day | day | day |
| Control | — | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| group | | | | | | | | | |
| Model | — | 9.2 ± 1.4 | 12.5 ± 1.8 | 13.6 ± 2.1 | 15.2 ± 1.7 | 17.4 ± 2.2 | 18.8 ± 2.1 | 18.6 ± 1.8 | 17.3 ± 1.4 |
| group | | | | | | | | | |
| Peptide | 174 | 7.3 ± 1.8* | 8.2 ± 1.5** | 9.5 ± 2.1** | 9.8 ± 1.7** | 10.2 ± 1.9** | 10.5 ± 1.9** | 11.4 ± 1.6** | 12.0 ± 1.6** |
| A high | | (20.6%) | (34.4%) | (30.1%) | (35.5%) | (41.4%) | (44.1%) | (38.7%) | (30.6%) |
| dose | | | | | | | | | |
| group | | | | | | | | | |
| Peptide | 87 | 8.2 ± 1.5 | 9.3 ± 2.2* | 9.9 ± 1.8** | 11.4 ± 1.6** | 12.2 ± 1.7** | 12.6 ± 2.1** | 13.7 ± 1.8** | 13.9 ± 1.7* |
| A | | (10.9%) | (25.6%) | (27.2%) | (25.0%) | (29.9%) | (33.0%) | (26.3%) | (19.6%) |
| medium | | | | | | | | | |
| group | | | | | | | | | |
| Peptide | 43.5 | 8.8 ± 1.6 | 10.2 ± 1.7* | 11.2 ± 1.8* | 12.3 ± 1.8* | 13.4 ± 1.7* | 14.6 ± 1.8* | 14.8 ± 2.2* | 14.5 ± 1.8* |
| A low | | (4.3%) | (18.4%) | (17.6%) | (19.1%) | (23.0%) | (22.3%) | (20.4%) | (16.2%) |
| dose | | | | | | | | | |
| group | | | | | | | | | |

Note:
Compared with the model group * P < 0.05, ** P < 0.01

3.2 Effect of Peptide A on Histopathological Examination Results for Rats with Combined Collagen-Adjuvant Induced Arthritis

The histopathological studies on combined collagen-adjuvant induced arthritis in rats showed that after induction by calf type II collagen in combination with Freund's complete adjuvant, joints of rats were manifested as synovial cell proliferation, the synovial cells were appeared to be nodular or clubbing and intruded into joint cavity when the proliferation was evident, and congestion of synovial tissue, inflammatory cell infiltration. After administration of the peptide A, the arthritis lesion was reduced in different degrees, as is shown in FIGS. 3a-5b.

It can be seen that treatment effects in descending order were as follows: peptide A high dose group, peptide A medium dose group, and peptide A low dose group. The treatment effects of these groups were significantly different from that of the model group. The each pathological index was comprehensively scored according to the scoring rules on lesion severity as described in the embodiment, using rank sum test method. The results were as shown in table 2.

TABLE 2
Scoring results of joint lesion severity

| | The number of | Lesion scoring |
|---|---|---|
| Group | the animal | ( X̅ ± SD) |
| Control group | 10 | 0.4 ± 0.32** |
| Model group | 10 | 6.32 ± 1.58 |
| Peptide A low dose group | 10 | 4.36 ± 1.15 |
| Peptide A medium dose group | 10 | 4.12 ± 1.28* |
| Peptide A high dose group | 10 | 3.73 ± 1.57* |

Compared with the model group:
*P < 0.05;
**P < 0.01

### Embodiment 2

### Protective Effect of Peptide A on Adjuvant Arthritis in Rats

1. Testing Material

1.1 Animal

SPF-level SD rats, each weighting 180 g-220 g, with male and female each in half, provided by Experimental Animal Center of Chinese Academy of Military Medical.

1.2 Pharmaceutical

Peptide A (which was prepared with the same procedure as in Embodiment 1). It was to be dissolved in physiological saline when used.

1.3 Preparation, does and Grouping of Pharmaceutical

SD rats, with male and female each in half, were randomly divided into five groups with 10 rats in each group, respectively were model group (employing physiological saline and Freund's complete adjuvant); control group (employing the same volume of physiological saline as the model group); peptide A high, medium and low dose groups (employing Freund's complete adjuvant and peptide A, wherein each group was given different doses of peptide A, respectively were 174 μg/kg·d, 87 μg/kg·d and 43.5 μg/kg·d, peptide A was dissolved in physiological saline to form peptide A solution with desired concentration). Peptide A solution in each dose was administered by subcutaneous injection, 0.1 ml of peptide A solution per 100 g body weight of rats. The control group employed the same amount of physiological saline as the peptide A solution (0.1 ml of physiological saline per 100 g body weight of rats).

2 Test Methods

2.1 Experimental Scheme

Each group rats were administered according to the following manner. These rats were normally fed every day during the administration.

On the 1st day (including this day), except the control group, for rats of other groups (the model group and the peptide A high, medium and low dose groups), 0.5 ml of Freund's complete adjuvant was subcutaneously injected into plantar skin of right rear foot of every rat. Then, they were subcutaneously injected with 0.05 ml of Freund's complete adjuvant for one time every other day, a total of 15 times.

From the 2nd day (including this day), the rats of peptide A high, medium and low dose groups each were administered with corresponding the dose of peptide A. Then, they were subcutaneously injected with the dose the amount of peptide A for one time every other day, a total of 15 times. At the same time, the rats of the control group and the model group were subcutaneously injected the same amount of physiological saline as that of the peptide A for the high, medium and low dose groups for one time every other day, a total of 15 times.

On the 13en day, varying grades of inflammatory reaction were observed from the rats of the model group, manifested as joints local heat, swelling, foot volume increase, lameness, etc. On the 24th day, for most rats of the model group, two rear foot joints and partial forefoot joints reached the greatest degree of swelling, and part of the rats had skin ulcers. Compared with rats of the model group, rats of peptides A high, medium and low dose groups were manifested as significantly reduced joint swelling, and they did not have skin ulcer symptom.

Measurement for the experimental results was conducted as follows. Firstly, before administration to rats of each group, left rear ankle circumference (rain) of each rat was measured by a flexible rule as a base value, and then circumference of left rear ankle with inflammation of each rat was measured at the 4th, 8th, 12th, 16th, 20th, 24th, 28th, and 32nd day, respectively. Swelling value obtained by circumference after inflammation minus circumference before inflammation was used to observe changes of primary and secondary inflammation of Freund's complete adjuvant-induced rats.

Secondly, at the 32nd day, rats of each group were killed, and their left rear foot ankle joints ware quickly taken out, fixed in 10% formalin and then embedded in paraffin after decalcification, sectioned and stained with HE. The proliferation of synovial tissue, degeneration of covering epithelial cells (synovial cells), congestion of interstitial substance, inflammatory cell infiltration, damage or fibrosis of arthrodial cartilage, and inflammatory cell infiltration or fibrosis of subcutaneous tissue around the joint, were observed. According to severity of joint lesions, the lesions were scored as “1 score (few or mild lesion)”, “2 scores (moderate or medium lesion)”, “3 scores (large or severe lesion)”, and “4 scores (very severe lesion)”. Very mild lesion was scored as “0.5”, and no lesion tissue was scored as “0”. Accumulate all scores to obtain a total score for one group, and then calculate average score ( X̅±SD) for each animal in each group. The lower score indicates the milder grade of the lesion.

2.2 Data processing: all of the data were processed, where the pathology score was conducted using rank sum test, and other data was processed using t test, and then the results were statistically analyzed.

3 Results

3.1 Effect of Peptide A on Adjuvant Arthritis in Rats

From the data shown in table 3 below that the effect of peptide A in different doses on ankle joint swelling value (mm) and inhibition rate (%) ( X̅±SD, n=10) of rats with adjuvant arthritis, it can be seen that Freund's complete adjuvant can cause significant joint swelling in rats. Compared to rats of the model group, rats of peptides A high, medium and low dose groups had significantly reduced joint inflammation reaction induced by Freund's complete adjuvant (*P<0.05 or **P<0.01), manifested as significantly reduced joint swelling value, where inhibition rate to the joint swelling in peptide A high dose group was nearly 40%.

TABLE 3

| | Dose | The 4^{th} | The 8^{th} | The 12^{th} | The 16^{th} | The 20^{th} | The 24^{th} | The 28^{th} | The 32^{nd} |
|---|---|---|---|---|---|---|---|---|---|
| Group | (μg/kg · d) | day | day | day | day | day | day | day | day |
| Control | — | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| group | | | | | | | | | |
| Model | — | 10.3 ± 1.5 | 14.2 ± 2.1 | 15.8 ± 1.6 | 18.4 ± 2.2 | 18.2 ± 1.8 | 17.5 ± 2.4 | 16.7 ± 1.4 | 15.8 ± 1.7 |
| group | | | | | | | | | |
| Peptide | 174 | 8.5 ± 1.5* | 10.2 ± 1.7** | 10.8 ± 1.5** | 11.1 ± 1.6** | 11.4 ± 1.8** | 10.8 ± 1.7** | 10.4 ± 1.8** | 9.5 ± 1.9** |
| A high | | (17.5%) | (28.2%) | (31.6%) | (39.7%) | (37.4%) | (38.3%) | (37.7%) | (39.9%) |
| dose | | | | | | | | | |
| group | | | | | | | | | |
| Peptide | 87 | 8.9 ± 1.6 | 11.9 ± 1.8* | 12.1 ± 2.3** | 12.9 ± 1.8** | 13.2 ± 1.4** | 13.0 ± 1.8** | 12.8 ± 1.6** | 12.5 ± 1.8* |
| A | | (13.6%) | (16.2%) | (23.4%) | (29.9%) | (27.5%) | (25.7%) | (23.4%) | (20.9%) |
| medium | | | | | | | | | |
| group | | | | | | | | | |
| Peptide | 43..5 | 9.3 ± 1.4 | 13.5 ± 2.1 | 13.9 ± 1.6 | 14.2 ± 1.7* | 14.6 ± 2.3* | 14.2 ± 1.4* | 14.0 ± 1.8* | 13.8 ± 1.5 |
| A low | | (9.7%) | (4.9%) | (12.0%) | (22.8%) | (19.8%) | (18.8%) | (16.2%) | (12.6%) |
| dose | | | | | | | | | |
| group | | | | | | | | | |

Note:
Compared with the model group *P < 0.05, **P < 0.01

3.2 Effect of Peptide A on Histopathological Examination Results for Rats with Adjuvant Arthritis

The histopathological studies on adjuvant arthritis in rats showed that after induction by Freund's complete adjuvant, joints of rats were manifested as proliferation, disorganization, and uneven surface of synovial cell, the synovial cells are appeared to be nodular or clubbing and intruded into joint cavity when the proliferation was evident, and congestion of synovial tissue, inflammatory cell infiltration. All synovial tissues showed no obvious fibrosis or ossification, and articular cartilage showed no obvious damage or fibrosis and other lesions. The dermis and subcutaneous tissue of the skin around the joint were infiltrated by inflammatory cell, mainly mononuclear macrophages, lymphocytes and a few neutrophils. The embodiments of the present invention showed that after administration of the peptide A, arthritis lesion was reduced in different degrees. It can be seen that treatment effects in descending order were as follows: peptide A high dose group, peptide A medium dose group, and peptide A low dose group. The treatment effects of these groups were significantly different from that of the model group. The each pathological index was comprehensively scored according to the scoring rules on lesion severity as described in the embodiment, using rank sum test method. The results were as shown in table 4.

TABLE 4
Scoring results of joint lesion severity

| | The number of | Lesion scoring |
|---|---|---|
| Group | the animal | ( X̅ ± SD) |
| Control group | 10 | 0.5 ± 0.53** |
| Model group | 10 | 5.10 ± 1.37 |
| Peptide A low dose group | 10 | 4.10 ± 0.99 |
| Peptide A medium dose group | 10 | 4.00 ± 0.67* |
| Peptide A high dose group | 10 | 3.60 ± 1.26* |

Compared with the model group:
*P < 0.05,
**P < 0.01

What is claimed is:

1. Use of a peptide represented by formula I or derivative thereof in the manufacture of a pharmaceutical for preventing or treating arthritis, the derivative comprising a pharmaceutically acceptable salt or ester of the peptide:
(formula I)
Xaa1-Gln-Xaa2-Xaa3-Thr-Ser-Gly-Xaa4
wherein,
Xaa1 is deleted, or Xaa1 is Ala, Gly, Val, Leu or Ile,
Xaa2 is Thr or Ser,
Xaa3 is Tyr, Phe or Trp, and
Xaa4 is deleted, or Xaa4 is Ala, Gly, Val, Leu, Ile or Pro.

2. Use according to claim 1, wherein the peptide or derivative thereof is a peptide represented by formula II or a pharmaceutically acceptable salt or ester thereof:
(formula II)
Gly-Gln-Thr-Tyr-Thr-Ser-Gly.

3. Use according to claim 1, wherein the pharmaceutical is in the form of unit formulation.

4. Use according to claim 1, wherein the pharmaceutical is in the form of injection formulation.

5. Use according to claim 1, wherein the arthritis is combined collagen-adjuvant induced arthritis.

6. Use according to claim 5, wherein the combined collagen-adjuvant induced arthritis is an arthritis induced by type II collagen in combination with Freund's complete adjuvant.

7. Use according to claim 1, wherein the arthritis is an adjuvant arthritis.

8. Use according to claim 7, wherein the adjuvant arthritis is an arthritis induced by Freund's complete adjuvant.

9. A method for treating arthritis, comprising administering a pharmaceutical containing therapeutically effective amount of the peptide of formula I or derivative thereof to a patient, where the derivative comprises a pharmaceutically acceptable salt or ester of the peptide:
(formula I)
Xaa1-Gln-Xaa2-Xaa3-Thr-Ser-Gly-Xaa4
wherein,
Xaa1 is deleted, or Xaa1 is Ala, Gly, Val, Leu or Ile,
Xaa2 is Thr or Ser,
Xaa3 is Tyr, Phe or Trp, and
Xaa4 is deleted, or Xaa4 is Ala, Gly, Val, Leu, Ile or Pro.

10. Method according to claim 9, wherein the peptide or derivative thereof is a peptide represented by formula II below or a pharmaceutically acceptable salt or ester thereof:
(formula II)
Gly-Gln-Thr-Tyr-Thr-Ser-Gly.

11. Method according to claim 9, comprising administering a pharmaceutical containing therapeutically effective amount of 300-3000 μg of the peptide or derivative thereof to a patient.

12. Method according to claim 11, comprising administering a pharmaceutical containing therapeutically effective amount of 480-1800 μg of the peptide or derivative thereof to a patient.

13. Method according to claim 9, wherein the pharmaceutical is administrated in the form of unit formulation to the patient.

14. Method according to claim 13, wherein the unit formulation comprises 500-3000 μg of the peptide or derivative thereof.

15. Method according to claim 14, wherein the unit formulation comprises 800-1800 μg of the peptide or derivative thereof.

16. Method according to claim 9, wherein the pharmaceutical is administrated by injection.

17. Method according to claim 9, wherein the arthritis is a combined collagen-adjuvant induced arthritis.

18. Method according to claim 17, wherein the combined collagen-adjuvant induced arthritis is an arthritis induced by type II collagen in combination with Freund's complete adjuvant.

19. Method according to claim 9, wherein the arthritis is an adjuvant arthritis.

20. Method according to claim 19, wherein the adjuvant arthritis is an arthritis induced by Freund's complete adjuvant.
